(19) United States
(12) Patent Application Publication
(10) Pub. No.: US 2024/0300870 A1
(43) Pub. Date: Sep. 12, 2024

(54) METHOD FOR OLIGOMERISATION IN A GAS/LIQUID REACTOR COMPRISING A CENTRAL DUCT

(71) Applicant: IFP ENERGIES NOUVELLES, Rueil-Malmaison (FR)

(72) Inventors: Ludovic RAYNAL, RUEIL-MALMAISON CEDEX (FR); Alexandre VONNER, RUEIL-MALMAISON CEDEX (FR); Pedro MAXIMIANO RAIMUNDO, RUEIL-MALMAISON CEDEX (FR)

(73) Assignee: IFP ENERGIES NOUVELLES, RUEIL-MALMAISON (FR)

(21) Appl. No.: 18/269,133

(22) PCT Filed: Dec. 14, 2021
(86) PCT No.: PCT/EP2021/085588

§ 371 Date: Jun. 22, 2023

(30) Foreign Application Priority Data
Dec. 23, 2020 (FR) 2014019

(51) Int. Cl. C07C 2/24; B01J 10/00; B01J 19/00; B01J 19/24

(52) U.S. Cl. CPC C07C 2/24; B01J 10/002; B01J 19/0013; B01J 19/2465; B01J 2219/0011; C07C 2521/06; C07C 2523/26; C07C 2523/755

(57) ABSTRACT

The present invention relates to a gas/liquid reactor for the oligomerization of gaseous ethylene, comprising a central pipe which delimits inside the reactor chamber a central zone allowing a descending flow and an outer zone allowing an ascending flow, thus making it possible to increase the time of travel of the injected gas bubbles in the liquid phase, without increasing the volume of the liquid phase and thus the volume of the reactor.

### TECHNICAL FIELD

The present invention relates to the field of gas/liquid reactors for the oligomerization of olefins to linear olefins by homogeneous catalysis.

The invention also relates to the use of the gas/liquid reactor in a process for the oligomerization of a gaseous olefinic feedstock, preferably of gaseous ethylene, to give linear α-olefins such as 1-butene, 1-hexene or 1-octene, or a mixture of linear α-olefins.

### PRIOR ART

The invention relates to the field of gas/liquid reactors, also known as bubble columns, and also to the use thereof in a process for the oligomerization of an olefinic feedstock, preferably of ethylene. One drawback encountered during the use of such reactors in ethylene oligomerization processes is the management of the gas headspace, corresponding to the upper part of the reactor in the gaseous state. Said gas headspace comprises gaseous compounds that are sparingly soluble in the liquid phase, compounds that are partially soluble in the liquid but which are inert, and also gaseous ethylene not dissolved in said liquid. The passage of gaseous ethylene from the liquid lower part of the reaction chamber to the gas headspace is a phenomenon referred to as breakthrough. In point of fact, the gas headspace is bled in order to remove said gaseous compounds. When the amount of gaseous ethylene present in the gas headspace is high, bleeding of the gas headspace leads to a not insignificant loss of ethylene, which is detrimental to the productivity and to the cost of the oligomerization process. Furthermore, a significant phenomenon of breakthrough means that a lot of gaseous ethylene has not been dissolved in the liquid phase and therefore has not been able to react, which is detrimental not only to the productivity but also to the selectivity of the oligomerization process.

In order to improve the efficiency of the oligomerization process notably in terms of productivity and of cost, it is thus essential to limit the phenomenon of breakthrough of the ethylene in order to improve its conversion in said process, while at the same time maintaining good selectivity for the desired linear α-olefins.

The prior art processes using a gas/liquid reactor, as illustrated in FIG. 1, do not make it possible to limit the loss of gaseous ethylene, and purging of the gas headspace results in an exit of gaseous ethylene from the reactor that is detrimental to the yield and the cost of the process.

In patent applications WO 2019/011806 and WO 2019/011609, the Applicant described processes enabling an increase in the contact surface area between the upper part of the liquid fraction and the gas headspace, by way of a dispersion means or vortex, so as to promote the passage of the ethylene contained in the gas headspace towards the liquid phase at the liquid/gas interface. These processes do not make it possible to limit the phenomenon of breakthrough and are insufficient when the amount of ethylene in the gas headspace is substantial because of a high level of breakthrough.

Furthermore, in the course of these research studies, the Applicant has found that in a reactor operating with a constant flow rate of injected gaseous ethylene, the amount of dissolved ethylene and thus the level of breakthrough are dependent on the dimensions of the reactors implementing the process, and notably on the height of the liquid phase, which conditions the dissolution time of the injected gas bubbles. The term “dissolution time” means the time between the moment when the bubble is injected and the moment when it disappears (total dissolution) or comes out of the liquid phase (breakthrough). This is because the lower the height, the shorter the time during which the gaseous ethylene travels through the liquid phase to become dissolved and the higher the level of breakthrough.

The Applicant has discovered that it is possible to improve the conversion of olefin(s), in particular of ethylene, while at the same time maintaining high selectivity towards desired linear olefin(s), and notably towards α-olefin(s), by limiting the phenomenon of breakthrough by means of a gas/liquid reactor for the oligomerization of gaseous ethylene, comprising a central pipe which delimits inside the reactor chamber a central zone allowing a descending flow and an outer zone allowing an ascending flow, thus making it possible to increase the time of travel of the injected gas bubbles in the liquid phase, without increasing the volume of the liquid phase and thus the volume of the reactor.

### BRIEF DESCRIPTION OF THE INVENTION

One subject of the present invention relates to a gas/liquid reactor for the oligomerization of a gaseous olefinic feedstock, preferably gaseous ethylene, comprising:
a reactor chamber 1, of elongated shape along a vertical axis,
a gas injection device 3,
a liquid injection device 11,
a central pipe 12 positioned on the vertical axis inside said chamber; said central pipe being immersed in a liquid phase and delimiting a central flow zone that is capable of permitting a descending flow and an outer flow zone that is capable of permitting an ascending flow,
   in which the gas injection device is positioned in the upper part of said central pipe and the liquid injection device is positioned in the reactor chamber so as to be able to entrain the injected gas in the direction of the lower part of the reactor, from the descending central zone to the ascending outer zone.

Preferably, the gas and liquid injection devices are positioned in the upper part of said central pipe so as to entrain the injected gaseous olefinic feedstock in the direction of the lower part of the reactor and from the descending central zone to the ascending outer zone. Preferably, the liquid injection device 11 is positioned above the gas injection device 3.

Preferably, the central pipe 12 has a solid wall over the entire height of the central pipe or has apertures over 5% to 10% of the lower part of the height of the central pipe from the lower end aperture.

Preferably, the lower part of the central pipe at the lower end aperture exhibits flaring or tapering.

Preferably, the central pipe comprises a deflector positioned in the reactor chamber and facing the lower end aperture of the central pipe. Preferably, the deflector is positioned at a distance with the lower aperture of the central pipe corresponding to a distance of between one and two times the equivalent diameter of the central pipe. Preferably, the equivalent diameter of the deflector is at least equal to the equivalent diameter of the central pipe and preferably between 0.5 and 2.0 times the diameter of the central pipe.

Preferably, the reactor comprises a recirculation loop comprising a withdrawing means located at the base of the reactor chamber, a heat exchanger located outside the reactor chamber and an introduction means located on or in the reactor chamber to allow the introduction of a cooled liquid fraction into the reactor chamber. Preferably, the liquid injection device 11 is positioned in the upper part of the central pipe and is connected to the introduction means of the recirculation loop.

Preferably, the central pipe has an equivalent diameter with a ratio of the equivalent diameter of the central pipe to the inside diameter of the reactor chamber of between 0.2 and 0.9, preferably between 0.3 and 0.8.

Preferably, the central pipe has a height with a ratio of the height of the central pipe to the height of the reactor chamber of between 0.2 and 0.8, preferably between 0.3 and 0.7.

Preferably, the gas injection device (3) comprises at least one gas injection orifice and the liquid injection device (11) comprises at least one liquid injection orifice, each gas injection orifice being positioned at an orifice of the liquid injection device (11) so that the injection of the liquid can bring about a reduction, by shear, of the size of the bubbles during the injection of the gaseous olefinic feedstock. Preferably, the gas injection orifices and the liquid injection orifices are extended by an injection tube.

Another subject of the invention relates to a process for the oligomerization of a gaseous olefinic feedstock, using a gas/liquid reactor as described above at a temperature of between 30 and 200° C. and a pressure of between 0.1 and 10.0 MPa, in the presence of a catalytic system comprising at least one metal precursor.

Preferably, the gaseous olefinic feedstock is preferably chosen from hydrocarbon-based molecules containing between 2 and 6 carbon atoms, preferably between 2 and 4 carbon atoms and in a preferred manner from butenes, more particularly isobutene or 1-butene, propylene and ethylene, alone or as a mixture.

### Definitions & Abbreviations

Throughout the description, the terms or abbreviations below have the following meaning.

The term “oligomerization” means any addition reaction of a first olefin to a second olefin, which may be identical to or different from the first olefin. The olefin thus obtained has the empirical formula CₙH₂ₙ, where n is equal to or greater than 4.

The term “linear α-olefin” means an olefin on which the double bond is located at the terminal position of the linear alkyl chain.

The term “catalytic system” means a chemical species which enables the use of the catalyst. The catalytic system may be a metal precursor comprising one or more metal atoms or a mixture of compounds for catalysing a chemical reaction, and more specifically an olefin oligomerization reaction. The mixture of compounds comprises at least one metal precursor. The mixture of compounds may also comprise an activator. The mixture of compounds may comprise an additive. The compound or the mixture of compounds may optionally be in the presence of a solvent.

The term “liquid phase” means the mixture of all of the compounds which are in a liquid physical state under the temperature and pressure conditions of the reaction chamber.

The term “gas phase” means the mixture of all of the compounds which are in the gaseous physical state under the temperature and pressure conditions of the reaction chamber: in the form of bubbles present in the liquid, and also in the top part of the reactor (or gas headspace of the reactor).

The terms “reactor chamber” and “reaction chamber” are used, without distinction between them, to denote the reactor chamber (1).

The term “lower zone of the reaction chamber” means the part of the chamber that comprises the liquid phase, the gaseous olefinic feedstock, in particular gaseous ethylene, the reaction products such as the desired linear α-olefin (i.e 1-butene, 1-hexene, 1-octene or the mixture of linear α-olefins), the catalytic system and optionally a solvent.

The term “upper zone of the reaction chamber” means the part of the chamber that is located at the apex of the chamber, i.e. directly above the lower zone and consisting of the gas phase corresponding to the gas headspace.

The term “uncondensable gas” means a species in gaseous physical form which only partially dissolves in the liquid under the temperature and pressure conditions of the reaction chamber and which can, under certain conditions, accumulate in the headspace of the reactor (for example here: ethane).

The terms “reactor” or “device” denote all of the means enabling the implementation of the oligomerization process according to the invention, notably such as the reaction chamber and the recirculation loop.

The term “lower part of the reaction chamber” means the lower quarter of the reaction chamber containing the liquid phase.

The term “upper part of the reaction chamber” means the upper quarter of the reaction chamber containing the liquid phase.

The expression “degree of saturation with dissolved gaseous olefinic feedstock, in particular dissolved ethylene” denotes the ratio of the amount of dissolved gaseous olefinic feedstock, in particular of dissolved ethylene, to the maximum amount of the dissolved gaseous olefinic feedstock, in particular of ethylene, which it is possible to dissolve in the liquid under the temperature and pressure conditions considered.

The term “equivalent diameter” is understood as being the diameter of the circle in which is inscribed the cross section (horizontal cross section) of the central pipe.

The various components of the reactor will be described with reference to all of the figures, each component retaining the same reference sign from one figure to another.

### BRIEF DESCRIPTION OF THE FIGURES

FIG. 1 illustrates a reactor according to the prior art. This reactor consists of a reactor chamber 1 comprising a lower zone comprising a liquid phase and an upper zone comprising a gas phase, a means 2 for introducing an olefinic feedstock such as gaseous ethylene, via a gas injection device 3, into the liquid phase. The upper part of the reaction chamber 1 comprising the gas phase comprises a bleed means 4. A pipe for the withdrawal of a liquid fraction 5 is located in the bottom of the reaction chamber 1. Said fraction 5 is divided into two streams, a first, main stream 7 which is sent to a heat exchanger 8 and then introduced via a pipe 9 into the liquid phase, and a second stream 6, which corresponds to the effluent sent to a later step. The pipe 10 in the bottom of the reaction chamber enables the introduction of the catalytic system.

FIG. 2 illustrates a reactor according to the invention which differs from the reactor of FIG. 1 in that the upper part of the lower zone of the chamber 1 comprises a central pipe 12 at the top of which are positioned the gas injection device 3 and a liquid injection device 11 so that the injection of the liquid brings about flow of the injected liquid and gas from the descending central flow zone to the ascending outer flow zone relative to the central pipe 12. The arrows represent the direction of circulation of the injected liquid and gas in the reactor chamber 1.

FIG. 3 illustrates another embodiment of the reactor according to the invention which differs from the reactor of FIG. 2 in that the liquid injection device 11 is connected to the pipe 9. Thus, the liquid stream exiting the heat exchanger 8 is injected at the top of the central pipe 12 via a liquid injection device 11 arranged with the gas injection device 3 so that the injection of the liquid brings about flow, towards the bottom of the reactor chamber, of the gas and of the liquid in the central pipe 12.

FIG. 4A is a schematic bottom view of a perpendicular cross section of the vertical axis of the reactor chamber of a preferred embodiment of the invention in which the gas injection device 3 and liquid injection device 11 are arranged so that the injection of the liquid can bring about, by shear, a reduction in the size of the gas bubbles of the injected gaseous olefinic feedstock by the injected liquid. The gas 3 and liquid 11 injection devices are of circular shape and arranged so that the outlet orifices 13 of the gas injection device 3 injects the gas towards the wall of the chamber 1 and so that the injection trajectory of the gas perpendicularly crosses the trajectory of the liquid injected via the orifices 14 so as to bring about shear of the gas in order to reduce the bubble size of the injected gas.

FIG. 4B is a schematic view of a vertical cross section along the vertical axis of the injection device of FIG. 4A. The liquid injection device 11 is a ring having a diameter greater than that of the gas injection device 3. The liquid injection device 11 is positioned on a plane above that of the gas injection device 3 so that each of the orifices 13 of the gas injection device 3 is positioned perpendicularly and on the side of one of the orifices 14 of the liquid injection device 11, so that the injected gas stream is on the trajectory of the stream of liquid injected at the orifices 14 of the liquid injection device 11.

FIG. 4C is a schematic view of a vertical cross section along the vertical axis of the injection devices according to FIG. 4A illustrating the effect of shear of the gas injected by the gas injection device 3 by the liquid injected by the liquid injection device 11.

### DETAILED DESCRIPTION OF THE INVENTION

It is specified that, throughout this description, the expression “between . . . and . . . ” should be understood as including the limits mentioned.

For the purposes of the present invention, the various embodiments presented may be used alone or in combination with each other, without any limit to the combinations.

For the purposes of the present invention, the various ranges of parameters for a given step, such as the pressure ranges and the temperature ranges, may be used alone or in combination. For example, for the purposes of the present invention, a range of preferred pressure values can be combined with a range of more preferred temperature values.

Throughout the description and in the claims, the positions (“bottom”, “top”, “above”, “below”, “horizontal”, “vertical”, “lower half”, etc.) of the various elements are defined relative to the column in the operating position.

The present invention relates to a gas/liquid reactor for the oligomerization of a gaseous olefinic feedstock, preferably gaseous ethylene, comprising:
a reactor chamber 1, of elongated shape along a vertical axis,
a gas injection device 3,
a liquid injection device 11,
a central pipe 12 positioned on the vertical axis in said chamber; said central pipe delimiting a descending central flow zone and an ascending outer flow zone,
   in which the gas injection device is positioned in the upper part of said central pipe and the liquid injection device is positioned in the reactor chamber so as to entrain the injected gas in the direction of the lower part of the reactor, from the descending zone to the ascending zone.

For the purposes of the present invention, the gas injection device is intended to inject an olefinic feedstock in gaseous form into an oligomerization reactor.

Advantageously, the reactor according to the invention makes it possible to increase the time for which the gaseous olefinic feedstock passes through the liquid phase, and thus to improve the dissolution of said feedstock in the liquid phase, which synergistically decreases the phenomenon of breakthrough. Another advantage of the reactor according to the invention is that the buoyancy exerted on the injected gaseous olefinic feedstock makes it possible to limit the speed of descent in the central pipe, which increases the time of travel of the gaseous olefinic feedstock in the liquid phase.

Advantageously, the degree of saturation with dissolved gaseous olefinic feedstock, in particular with dissolved ethylene, in the liquid phase is greater than 70.0%, preferably between 70.0% and 100%, preferably between 80.0% and 100%, preferably between 80.0% and 99.0%, preferably between 85.0% and 99.0% and even more preferably between 90.0% and 98.0%.

The degree of saturation with dissolved ethylene can be measured by any method known to those skilled in the art, for example by gas chromatography (commonly referred to as GC) analysis of a fraction of the liquid phase withdrawn from the reaction chamber.

Another advantage of the present invention is that it improves the conversion of the olefinic feedstock, in particular of ethylene, and/or the selectivity in particular for α-olefins, and also the volumetric productivity of the oligomerization process.

Another advantage of the reactor according to the invention is that it makes it possible to reduce the reaction volume and thus the dimensions of the reactor relative to a reactor according to the prior art, for identical performance.

### Reactor

The present invention relates to a process for the oligomerization of a gaseous olefinic feedstock, at a temperature of between 30 and 200° C., and a pressure of between 0.1 and 10.0 MPa, in the presence of a catalytic system comprising at least one metal precursor, said process employing a gas/liquid reactor for the oligomerization of a gaseous olefinic feedstock, preferably gaseous ethylene, comprising
a reactor chamber (1), of elongated shape along a vertical axis,
a gas injection device (3),
a liquid injection device (11),
a central pipe (12) positioned on the vertical axis inside said chamber in a lower zone of said chamber; said central pipe delimiting a central flow zone that is capable of permitting a descending flow and an outer flow zone that is capable of permitting an ascending flow, in which the gas injection device is positioned in the upper part of said central pipe and the liquid injection device is positioned in the lower zone of the reactor chamber so as to be able to entrain the injected gas in the direction of the lower part of the reactor, from the descending central zone to the ascending outer zone.

In a preferred embodiment, the gas and liquid injection devices are positioned in the upper part of said central pipe and preferably close to each other, so as to advantageously entrain the injected gaseous olefinic feedstock in the direction of the lower part of the reactor and from the central zone to the outer zone. In this embodiment, the liquid injection device 11 is positioned above the gas injection device 3 so as to improve the entrainment of the gas corresponding to the gaseous olefinic feedstock by the liquid in the direction of a descending flow in the central pipe.

In another embodiment, the liquid injection device is positioned in the ascending zone between the reactor chamber and the central pipe so as to entrain the injected gaseous olefinic feedstock in the direction of the lower part of the reactor from the descending zone to the ascending zone.

Preferably, the central pipe 12 is positioned substantially at the centre of the reactor chamber on the vertical axis in said chamber.

Preferably, the oligomerization reactor is a reactor for the dimerization, trimerization or tetramerization of, for example, ethylene.

The combination of the liquid injection 11 and gas injection 3 devices and of the central pipe 12, when the reactor is implemented in an oligomerization process, makes it possible to increase the residence time during which the gaseous olefinic feedstock remains in the liquid phase, before possibly joining the gas headspace, which improves the dissolution of the gaseous olefinic feedstock, in particular gaseous ethylene, in said liquid phase.

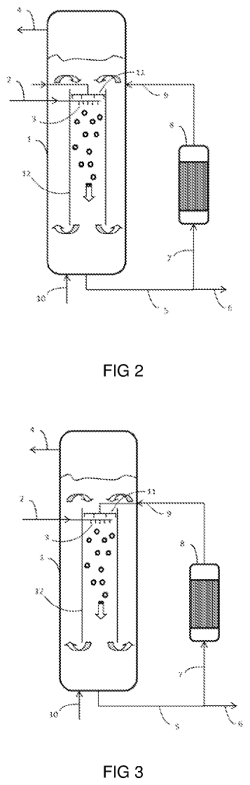

Thus, according to the invention, the lower end and the upper end of the central pipe 12 are open so as to allow free circulation of and to orient the circulation of the liquid in the reactor chamber 1, as illustrated in FIG. 2. The injection of liquid, preferably in the upper part of the central pipe, is performed so as to direct the flow of the gas and of the liquid in a descending flow inside the central pipe, and an ascending flow outside the central pipe.

The central pipe may advantageously have a circular, oval, triangular or square cross section or any other geometrical shape that is suitable for the implementation of the reactor according to the invention. Preferably, the central pipe has a circular cross section. Advantageously, the cross section is identical over the entire height of the pipe.

It is understood that the central pipe, and also the gas and liquid injection devices, are positioned in a lower zone so as to be immersed in the liquid phase when the reactor according to the invention is implemented in a process for the oligomerization of a gaseous olefinic feedstock.

In a particular embodiment, the central pipe 12 has a solid wall over the entire height of the central pipe or has apertures over 5% to 10% of the lower part of the height of the central pipe from the lower end aperture.

Preferably, the lower part of the central pipe at the lower end aperture exhibits flaring or tapering.

In a preferred embodiment, the central pipe also comprises a deflector positioned facing the lower end aperture. Preferably, said deflector is positioned at a distance with the lower aperture of the central pipe corresponding to a distance of between one and two times the equivalent diameter of the central pipe. Preferably, the deflector may have any shape, for example a circular or oval disc, and may advantageously be solid or may comprise holes. Advantageously, said holes may be of round or oval shape or alternatively rectangular slits. Preferably, the central pipe is of cylindrical shape, the deflector is of cylindrical shape and the diameter of said deflector is at least equal to the diameter of the central pipe, preferably between 0.5 and 2.0 and preferably between 1.0 and 1.5 times the diameter of the central pipe.

Advantageously, irrespective of the embodiment, the integral fastening of the central pipe and/or of the optional deflector in the reactor chamber is performed, for example, by means of lugs, girders or any other rigid structure, connecting the various elements to be assembled, such as the central pipe wall and the reactor chamber, said lugs possibly being fixed by welding, by bonding, by screwing or by bolting, alone or in combination, or any other similar means. In particular, the integral fastening of the central pipe and of the reactor chamber wall is performed so as to release a passage section corresponding to the ascending outer zone.

Preferably, the reactor also comprises a recirculation loop comprising a withdrawing means located at the base (preferably at the bottom) of the reactor chamber, a heat exchanger advantageously located outside the reactor chamber, and an introduction means advantageously located on or in the reactor chamber to allow the introduction of a cooled liquid fraction into the reactor chamber. Thus, when the reactor is implemented in an oligomerization process and when the recirculation loop is functioning, a liquid fraction is withdrawn from the reactor chamber and sent to the heat exchanger to cool said withdrawn liquid fraction, which is subsequently introduced into the reactor via the introduction means.

In a preferred embodiment, the liquid injection device 11 is positioned in the upper part of the central pipe and is connected to the introduction means of the recirculation loop. Thus, the cooled liquid may be advantageously injected into said central pipe. One advantage of this embodiment is that the stream of injected cooled liquid participates in the entrainment of the olefinic feedstock, preferably ethylene, towards the bottom of the central pipe from the descending central zone to the ascending outer zone.

Another advantage of this embodiment is that it limits the material investment by maximizing the use of the recirculation loop and thus limits the overall cost of the oligomerization reactor.

Another advantage is that the liquid coming from the recirculation loop and introduced via the liquid injection device is colder and contains less ethylene than the liquid phase contained in the reactor. These two characteristics make it possible to improve the dissolution of the gaseous ethylene in the cooled liquid fraction.

Advantageously, the descending central zone inside the central pipe may comprise structured packing, of static mixer type or any other equivalent equipment which generates good stirring of the gas liquid flow, over a portion or all of its height, thus enabling better dissolution of the gas in the liquid via the turbulence generated by the structured packing.

Preferably, the reactor chamber 1 is cylindrical. In the case of a cylindrical chamber, the diameter D is the diameter of the cylinder. Such a geometry makes it possible notably to limit the presence of “dead” volumes in the column.

Preferably, the central pipe has an equivalent diameter with a ratio of the equivalent diameter of the central pipe to the inside diameter of the reactor chamber of between 0.2 and 0.9, preferably between 0.3 and 0.8. In the case where the central pipe is of cylindrical shape, the equivalent diameter of the pipe corresponds to the diameter of the cross section (horizontal cross section) of the central pipe.

Preferably, the central pipe has a height with a ratio of the height of the central pipe to the height of the reactor chamber of between 0.2 and 0.8, preferably between 0.3 and 0.7. In particular, the ratio of the height of the central pipe to the height of the reactor chamber is equal to 0.2, 0.3, 0.4, 0.5 or 0.6.

Preferably, the reactor chamber 1 is of elongated shape along the vertical axis and may contain a liquid phase located in a lower zone comprising, and preferably consisting of, reaction products, dissolved and gaseous ethylene, a catalytic system and an optional solvent, and a gas phase (or gas headspace) located in an upper zone above the lower zone, comprising a fraction of the gaseous olefinic feedstock, preferably gaseous ethylene, and also uncondensable gases (notably ethane).

In particular, the gas/liquid reactor also comprises:
a means for introducing the catalytic system, said catalytic system comprising a metal catalyst, at least one activator and at least one additive, said means optionally being located in the lower part of the reactor chamber,
a means for withdrawing liquid phase to recover a reaction effluent comprising the produced α-olefins,
optionally a system for bleeding the gas headspace,
and optionally a gas phase recycling loop to recycle at least a fraction of the gas phase into the lower zone of the liquid phase, comprising a withdrawing means located in the upper zone of the reactor chamber to enable the withdrawal of a gas fraction in the gas phase and an introduction means in the lower zone of the reactor chamber to enable the introduction of said withdrawn gas fraction into the liquid phase.

Advantageously, the central pipe is positioned in the reactor chamber in the upper part of the lower zone, i.e. of the zone intended to contain the liquid phase, and preferably at a distance from the bottom of the reactor chamber that is suitable for enabling circulation of the liquid and gas streams.

Preferably, the gas injection device 3 is chosen from a pipe, a network of pipes, a multitubular distributor, a perforated plate, concentric tubes or any other means known to a person skilled in the art.

Preferably, the liquid injection device 11 is chosen from a pipe, a network of pipes, a multitubular distributor, a perforated plate, concentric tubes or any other means known to a person skilled in the art.

In a preferred embodiment, the gas injection device 3 comprises at least one gas injection orifice and the liquid injection device 11 comprises at least one liquid injection orifice, each gas injection orifice being positioned relative to at least one orifice of the liquid injection device 11, in particular in the upper part of the central pipe, so that the injection of the liquid can bring about a reduction, by shear, of the size of the bubbles during the injection of the gaseous olefinic feedstock. Thus, the injection trajectory of the gas is advantageously in the plane of the injection trajectory of the liquid. In this configuration, the injection of the liquid can then bring about the shear of the injected gas and result in a decrease in the size of the gas bubbles, making it possible to improve the dissolution of the gas in the liquid phase by increasing the interface between the gas and the liquid.

It is understood that the gas and liquid injection devices may comprise a plurality of injection orifices as a function of the dimensions of the reactor insofar as said injection devices are arranged so that the injection of the liquid can bring about a reduction, by shear, of the bubble size during the injection of the gaseous olefinic feedstock.

Advantageously, the arrangement according to this preferred embodiment makes it possible to reduce the size of the injected gas bubbles by at least 20% relative to the size of the injected gas bubbles without shear. Preferably, the percentage decrease in the size of the bubbles by this shear is at least 25% relative to the size of the injected gas bubbles without shear, preferably at least 30%, preferably at least 35% and in a preferred manner at least 40%.

Advantageously, the breakdown of a gas bubble into two smaller bubbles of the same size gives rise to a 26% increase in the area of exchange between the gas and the liquid, the breakdown of a gas bubble into four smaller bubbles of the same size gives rise to a 59% increase, and the breakdown of a gas bubble into six smaller bubbles of the same size gives rise to an 82% increase. Hence, a reactor according to the invention facilitates and thus significantly improves the absorption of gas in the liquid phase, which makes it possible to increase the saturation with gaseous olefinic feedstock in the liquid phase and to limit the phenomenon of breakthrough.

The term “injection orifice” means a round hole, an oval hole, a slit or any other form for injecting the liquid or the gas into the reactor. Preferably, the gas injection and liquid injection orifices are circular, i.e. round holes.

Preferably, the gas injection orifices have a diameter of between 1.0 and 15.0 mm, preferably between 3.0 and 20.0 mm, in order to form ethylene bubbles of millimetric size in the liquid. Preferably, the liquid injection orifices have a diameter of between 1.0 and 15.0 mm, preferably between 3.0 and 20.0 mm. Preferably, the liquid injection orifices have a diameter greater than or equal to the diameter of the gas injection orifices. Preferably, the ratio between the diameter of a gas injection orifice and the diameter of the liquid injection orifice arranged close to said gas injection orifice is between 0.1 and 1.0, preferably between 0.4 and 0.8.

In a preferred embodiment, the orifices of the gas and liquid injection devices are extended by a tube. Preferably, the tube of the gas injection device 13 have a diameter smaller than that of the tube of the liquid injection device 15 and is positioned coaxially inside the liquid injection tube. The outlet orifice of the gas injection tube is directed towards the outlet orifice of the liquid injection tube.

Preferably, the liquid injection tube 15 comprises a deflector as a means for partial closure of the tube, preferably a circular, round or square plate, which may or may not be perforated.

Advantageously, the deflector makes it possible to improve the effect of shear of the gas bubbles by the liquid.

Preferably, the end of the liquid injection tube has tapering of the outlet diameter. Said tapering brings about acceleration of the gas-liquid mixture, which makes it possible to increase the shear forces and further improves the breakdown of the gas bubbles into gas bubbles of smaller size.

In a very preferred embodiment, the tube has tapering of the outlet diameter and a deflector.

Advantageously, a gas injection orifice and a liquid injection orifice are positioned facing each other at an angle of between 0° and 180°. When the orifices of the gas and liquid injection devices are extended by a tube, the gas and liquid injection orifices correspond to the outlet orifices of the gas and liquid injection tube(s). An angle of 0° means that the gas and the liquid are injected via said respective orifices on the same trajectory axis and in the same direction. Preferably, the angle formed by the trajectories is between 0° and 120°, preferably between 30° and 120° and preferably between 45° and 90°. Very preferably, the angle formed by the trajectories is between 0° and 90°. Preferably, the angle formed by the trajectories is equal to 0°, 30°, 45°, 90°, 120° or 180°.

In a particular embodiment, the gas injection device is a cylindrical tube having a circular ring shape, for example round or oval, and having injection orifices. Advantageously, the liquid injection device is also a cylindrical tube having a circular ring shape, for example round or oval, and having injection orifices. Preferably, said liquid injection device is positioned in the upper part of said central pipe, close to said gas injection device and such that one (preferably each) gas injection orifice is positioned close to an orifice of the liquid injection device 11 so that the injection trajectory of the liquid is in the same plane as the injection trajectory of the gas so as to bring about the shear of said gas.

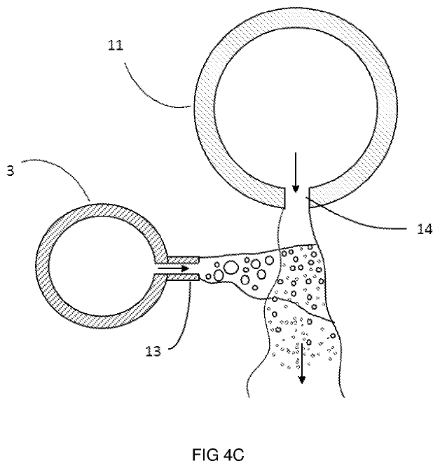

Advantageously, the gas injection device is in ring form and has a diameter greater or less than that of the liquid injection device in ring form. When the diameter of the gas injection device is less than that of the liquid injection device, the gas injection device is positioned inside the liquid injection device, as illustrated in FIG. 4A, on a different plane, i.e. above or below, preferably below (the liquid injection device then being found above the gas injection device). Conversely, when the diameter of the gas injection device is greater than that of the liquid injection device, the gas injection device is positioned outside the liquid injection device on a different plane, i.e. above or below.

In a particular embodiment, a sequence of several liquid and gas injection devices of circular form with decreasing diameters are alternated from the periphery to the centre represented by the central axis of the injection device having the largest diameter. Said devices are positioned so that a gas injection orifice of a gas injection device is positioned close to and facing an orifice of the adjacent liquid injection device so that the injection trajectory of the liquid is in the same plane as the injection trajectory of the gas so as to bring about the shear of said gas.

### Oligomerization Process

Another subject of the invention relates to the process for the oligomerization of a gaseous olefinic feedstock, preferably gaseous ethylene, using a gas/liquid reactor according to the invention as defined above.

Preferably, said process comprises the contact of a liquid and of the gaseous olefinic feedstock, preferably gaseous ethylene, by means of a gas injection device and of a liquid injection device, said gas and liquid injection devices being positioned in the upper part of a central pipe located in the reactor chamber, so as to entrain the injected gas in the direction of the lower part of the reactor, and then from the descending zone to the ascending zone.

Preferably, the injection speed of the liquid is greater than the injection speed of the gaseous olefinic feedstock so as to promote shear of the injected bubbles of the gaseous olefinic feedstock to gas bubbles of smaller size.

The gaseous olefinic feedstock is preferably chosen from hydrocarbon-based molecules containing between 2 and 6 carbon atoms, preferably between 2 and 4 carbon atoms. Preferably, the olefinic feedstock is chosen from butene, more particularly isobutene or 1-butene, propylene and ethylene, alone or as a mixture.

Preferably, the oligomerization process is a process for the dimerization, trimerization or tetramerization of, for example, ethylene.

The process for the oligomerization of a gaseous olefinic feedstock using the reactor according to the invention makes it possible to produce linear α-olefins by placing said olefinic feedstock in contact with a catalytic system, optionally in the presence of a solvent.

All the catalytic systems known to those skilled in the art and capable of being employed in dimerization, trimerization or tetramerization processes and more generally in the oligomerization processes according to the invention come within the field of the invention. Said catalytic systems and also the implementations thereof are notably described in patent applications FR 2 984 311, FR 2 552 079, FR 3 019 064, FR 3 023 183, FR 3 042 989 or else in patent application FR 3 045 414.

Preferably, the catalytic systems comprise, and preferably consist of:
a metal precursor, preferably based on nickel, titanium or chromium,
optionally an activating agent,
optionally an additive, and
optionally a solvent.

### The Metal Precursor

The metal precursor used in the catalytic system is chosen from compounds based on nickel, titanium or chromium.

In one embodiment, the metal precursor is based on nickel and preferentially comprises nickel in (+11) oxidation state. Preferably, the nickel precursor is chosen from nickel(II) carboxylates, for instance nickel 2-ethylhexanoate, nickel(II) phenates, nickel(II) naphthenates, nickel(II) acetate, nickel(II) trifluoroacetate, nickel(II) triflate, nickel(II) acetylacetonate, nickel(II) hexafluoroacetylacetonate, π-allylnickel(II) chloride, π-allylnickel(II) bromide, methallylnickel(II) chloride dimer, η³-allylnickel(II) hexafluorophosphate, η³-methallylnickel(II) hexafluorophosphate and nickel(II) 1,5-cyclooctadienyl, in their hydrated or non-hydrated form, taken alone or as a mixture.

In a second embodiment, the metal precursor is based on titanium and preferentially comprises a titanium aryloxy or alkoxy compound.

The titanium alkoxy compound advantageously corresponds to the general formula [Ti(OR)₄] in which R is a linear or branched alkyl radical. Among the preferred alkoxy radicals, non-limiting examples that may be mentioned include tetraethoxy, tetraisopropoxy, tetra(n-butoxy) and tetra(2-ethylhexyloxy).

The titanium aryloxy compound advantageously corresponds to the general formula [Ti(OR′)₄] in which R′ is an aryl radical which is unsubstituted or substituted with alkyl or aryl groups. The radical R′ may include heteroatom-based substituents. The preferred aryloxy radicals are chosen from phenoxy, 2-methylphenoxy, 2,6-dimethylphenoxy, 2,4,6-trimethylphenoxy, 4-methylphenoxy, 2-phenylphenoxy, 2,6-diphenylphenoxy, 2,4,6-triphenylphenoxy, 4-phenylphenoxy, 2-(tert-butyl)-6-phenylphenoxy, 2,4-di(tert-butyl)-6-phenylphenoxy, 2,6-diisopropylphenoxy, 2,6-di(tert-butyl)phenoxy, 4-methyl-2,6-di(tert-butyl)phenoxy, 2,6-dichloro-4-(tert-butyl)phenoxy and 2,6-dibromo-4-(tert-butyl)phenoxy, the biphenoxy radical, binaphthoxy and 1,8-naphthalenedioxy.

According to a third embodiment, the metal precursor is based on chromium and preferentially comprises a chromium(II) salt, a chromium(III) salt or a salt of different oxidation state which may include one or more identical or different anions, for instance halides, carboxylates, acetylacetonates or alkoxy or aryloxy anions. Preferably, the chromium-based precursor is chosen from CrCl₃, CrCl₃(tetrahydrofuran)₃, Cr(acetylacetonate)₃, Cr(naphthenate)₃, Cr(2-ethylhexanoate)₃ and Cr(acetate)₃.

The concentration of nickel, titanium or chromium is between 0.001 and 300.0 ppm by mass of atomic metal, relative to the reaction mass, preferably between 0.002 and 100.0 ppm, preferentially between 0.003 and 50.0 ppm, more preferentially between 0.05 and 20.0 ppm and even more preferentially between 0.1 and 10.0 ppm by mass of atomic metal, relative to the reaction mass.

### The Activating Agent

Optionally, irrespective of the metal precursor, the catalytic system comprises one or more activating agents chosen from aluminium-based compounds, such as methylaluminium dichloride (MeAlCl₂), dichloroethylaluminium (EtAlCl₂), ethylaluminium sesquichloride (Et₃Al₂Cl₃), chlorodiethylaluminium (Et₂AlCl), chlorodiisobutylaluminium (i-Bu₂AlCl), triethylaluminium (AlEt₃), tripropylaluminium (Al(n-Pr)₃), triisobutylaluminium (Al(i-Bu)₃), diethylethoxyaluminium (Et₂AlOEt), methylaluminoxane (MAO), ethylaluminoxane and modified methylaluminoxanes (MMAO).

### The Additive

Optionally, the catalytic system comprises one or more additives.

The additive is chosen from monodentate phosphorus-based compounds, bidentate phosphorus-based compounds, tridentate phosphorus-based compounds, olefinic compounds, aromatic compounds, nitrogenous compounds, bipyridines, diimines, monodentate ethers, bidentate ethers, monodentate thioethers, bidentate thioethers, monodentate or bidentate carbenes, mixed ligands such as phosphinopyridines, iminopyridines, bis(imino)pyridines.

When the catalytic system is based on nickel, the additive is preferably chosen from:
compounds of nitrogenous type, such as trimethylamine, triethylamine, pyrrole, 2,5-dimethylpyrrole, pyridine, 2-methylpyridine, 3-methylpyridine, 4-methylpyridine, 2-methoxypyridine, 3-methoxypyridine, 4-methoxypyridine, 2-fluoropyridine, 3-fluoropyridine, 3-trifluoromethylpyridine, 2-phenylpyridine, 3-phenylpyridine, 2-benzylpyridine, 3,5-dimethylpyridine, 2,6-di(tert-butyl)pyridine and 2,6-diphenylpyridine, quinoline, 1,10-phenanthroline, N-methylpyrrole, N-butylpyrrole, N-methylimidazole, N-butylimidazole, 2,2′-bipyridine, N,N′-dimethylethane-1,2-diimine, N,N′-di(t-butyl)ethane-1,2-diimine, N,N′-di(t-butyl)butane-2,3-diimine, N,N′-diphenylethane-1,2-diimine, N,N′-bis(2,6-dimethylphenyl)ethane-1,2-diimine, N,N′-bis(2,6-diisopropylphenyl)ethane-1,2-diimine, N,N′-diphenylbutane-2,3-diimine, N,N′-bis(2,6-dimethylphenyl)butane-2,3-diimine or N,N′-bis(2,6-diisopropylphenyl)butane-2,3-diimine, or
compounds of phosphine type independently chosen from tributylphosphine, triisopropylphosphine, tricyclopentylphosphine, tricyclohexylphosphine, triphenylphosphine, tris(o-tolyl)phosphine, bis(diphenylphosphino)ethane, trioctylphosphine oxide, triphenylphosphine oxide or triphenyl phosphite, or
compounds corresponding to the general formula (1) or one of the tautomers of said compound:

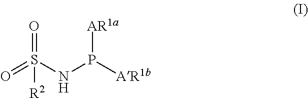

in which:
A and A′, which may be identical or different, are independently an oxygen or a single bond between the phosphorus atom and a carbon atom,
the groups R^{1a} and R^{1b} are independently chosen from methyl, trifluoromethyl, ethyl, n-propyl, isopropyl, n-butyl, isobutyl, t-butyl, pentyl, cyclohexyl and adamantyl groups, which may or may not be substituted and may or may not contain heteroelements; phenyl, o-tolyl, m-tolyl, p-tolyl, mesityl, 3,5-dimethylphenyl, 4-(n-butyl)phenyl, 2-methylphenyl, 4-methoxyphenyl, 2-methoxyphenyl, 3-methoxyphenyl, 4-methoxyphenyl, 2-isopropoxyphenyl, 4-methoxy-3,5-dimethylphenyl, 3,5-bis(tert-butyl)-4-methoxyphenyl, 4-chlorophenyl, 3,5-bis(trifluoromethyl)phenyl, benzyl, naphthyl, bisnaphthyl, pyridyl, bisphenyl, furyl and thiophenyl groups,
the group R² is independently chosen from methyl, trifluoromethyl, ethyl, n-propyl, isopropyl, n-butyl, isobutyl, t-butyl, pentyl, cyclohexyl and adamantyl groups, which may or may not be substituted and may or may not contain heteroelements; phenyl, o-tolyl, m-tolyl, p-tolyl, mesityl, 3,5-dimethylphenyl, 4-(n-butyl)phenyl, 4-methoxyphenyl, 2-methoxyphenyl, 3-methoxyphenyl, 4-methoxyphenyl, 2-isopropoxyphenyl, 4-methoxy-3,5-dimethylphenyl, 3,5-di(tert-butyl)-4-methoxyphenyl, 4-chlorophenyl, 3,5-bis(trifluoromethyl)phenyl, benzyl, naphthyl, bisnaphthyl, pyridyl, bisphenyl, furyl and thiophenyl groups.

When the catalytic system is based on titanium, the additive is preferably chosen from diethyl ether, diisopropyl ether, dibutyl ether, diphenyl ether, 2-methoxy-2-methylpropane, 2-methoxy-2-methylbutane, 2,2-dimethoxypropane, 2,2-bis(2-ethylhexyloxy)propane, 2,5-dihydrofuran, tetrahydrofuran, 2-methoxytetrahydrofuran, 2-methyltetrahydrofuran, 3-methyltetrahydrofuran, 2,3-dihydropyran, tetrahydropyran, 1,3-dioxolane, 1,3-dioxane, 1,4-dioxane, dimethoxyethane, bis(2-methoxyethyl) ether, benzofuran, glyme and diglyme, taken alone or as a mixture.

When the catalytic system is based on chromium, the additive is preferably chosen from:
compounds of nitrogenous type, such as trimethylamine, triethylamine, pyrrole, 2,5-dimethylpyrrole, pyridine, 2-methylpyridine, 3-methylpyridine, 4-methylpyridine, 2-methoxypyridine, 3-methoxypyridine, 4-methoxypyridine, 2-fluoropyridine, 3-fluoropyridine, 3-trifluoromethylpyridine, 2-phenylpyridine, 3-phenylpyridine, 2-benzylpyridine, 3,5-dimethylpyridine, 2,6-di(tert-butyl)pyridine and 2,6-diphenylpyridine, quinoline, 1,10-phenanthroline, N-methylpyrrole, N-butylpyrrole, N-methylimidazole, N-butylimidazole, 2,2′-bipyridine, N,N′-dimethylethane-1,2-diimine, N,N′-di(t-butyl)ethane-1,2-diimine, N,N′-di(t-butyl)butane-2,3-diimine, N,N′-diphenylethane-1,2-diimine, N,N′-bis(2,6-dimethylphenyl)ethane-1,2-diimine, N,N′-bis(2,6-diisopropylphenyl)ethane-1,2-diimine, N,N′-diphenylbutane-2,3-diimine, N,N′-bis(2,6-dimethylphenyl)butane-2,3-diimine or N,N′-bis(2,6-diisopropylphenyl)butane-2,3-diimine, or
aryloxy compounds of general formula [M(R³O)₂₋ₙXₙ]_{y}, in which:
M is chosen from magnesium, calcium, strontium and barium, preferably magnesium,
R³ is an aryl radical containing from 6 to 30 carbon atoms and X is a halogen or an alkyl radical containing from 1 to 20 carbon atoms,
n is an integer which can take the values of 0 or 1, and
y is an integer between 1 and 10; preferably, y is equal to 1, 2, 3 or 4.

Preferably, the aryloxy radical R³O is chosen from 4-phenylphenoxy, 2-phenylphenoxy, 2,6-diphenylphenoxy, 2,4,6-triphenylphenoxy, 2,3,5,6-tetraphenylphenoxy, 2-(tert-butyl)-6-phenylphenoxy, 2,4-di(tert-butyl)-6-phenylphenoxy, 2,6-diisopropylphenoxy, 2,6-dimethylphenoxy, 2,6-di(tert-butyl)phenoxy, 4-methyl-2,6-di(tert-butyl)phenoxy, 2,6-dichloro-4-(tert-butyl)phenoxy and 2,6-dibromo-4-(tert-butyl)phenoxy. The two aryloxy radicals may be borne by the same molecule, for instance the biphenoxy radical, binaphthoxy or 1,8-naphthalenedioxy. Preferably, the aryloxy radical R³O is 2,6-diphenylphenoxy, 2-(tert-butyl)-6-phenylphenoxy or 2,4-di(tert-butyl)-6-phenylphenoxy.

### The Solvent

In another embodiment according to the invention, the catalytic system optionally comprises one or more solvents.

In one embodiment, a solvent or a mixture of solvents may be used during the oligomerization reaction.

The solvent(s) are advantageously chosen from ethers, alcohols, halogenated solvents and hydrocarbons, which may be saturated or unsaturated, cyclic or non-cyclic, aromatic or non-aromatic, comprising between 1 and 20 carbon atoms, preferably between 4 and 15 carbon atoms, preferentially between 4 and 12 carbon atoms and even more preferentially between 4 and 8 carbon atoms.

Preferably, the solvent is chosen from pentane, hexane, cyclohexane, methylcyclohexane, heptane, butane or isobutane, 1,5-cyclooctadiene, benzene, toluene, ortho-xylene, mesitylene, ethylbenzene, diethyl ether, tetrahydrofuran, 1,4-dioxane, dichloromethane, dichloroethane, tetrachloroethane, hexachloroethane, chlorobenzene, dichlorobenzene, butene, hexene and octene, pure or as a mixture.

Preferably, the solvent may be advantageously chosen from the products of the oligomerization reaction. Preferably, the solvent used is cyclohexane.

Preferably, when a solvent is used in the oligomerization process, the mass content of solvent introduced into the reactor used in the process according to the invention is between 0.2 and 10.0, preferably between 0.5 and 5.0, and in a preferred manner between 1.0 and 4.0. The content of solvent is the mass ratio of the total flow rate of injected solvent to the total flow rate of injected gaseous ethylene in the process.

Preferably, the linear α-olefins obtained comprise from 4 to 20 carbon atoms, preferably from 4 to 18 carbon atoms, preferably from 4 to 10 carbon atoms and preferably from 4 to 8 carbon atoms. Preferably, the olefins are linear α-olefins chosen from 1-butene, 1-hexene and 1-octene.

Advantageously, the oligomerization process is performed at a pressure of between 0.1 and 10.0 MPa, preferably between 0.2 and 9.0 MPa and preferentially between 0.3 and 8.0 MPa, at a temperature of between 30 and 200° C., preferably between 35 and 150° C. and in a preferred manner between 45 and 140° C.

Preferably, the concentration of catalyst in the catalytic system is between 0.001 and 300.0 ppm by mass of atomic metal relative to the reaction mass, preferably between 0.002 and 100.0 ppm, preferentially between 0.003 and 50.0 ppm, more preferentially between 0.05 and 20.0 ppm and even more preferentially between 0.1 and 10.0 ppm by mass of atomic metal relative to the reaction mass.

According to one embodiment, the oligomerization process is performed batchwise. The catalytic system, constituted as described above, is introduced into a reactor according to the invention, advantageously equipped with heating and cooling devices, then pressurization with ethylene is performed to the desired pressure, and the temperature is adjusted to the desired value. The pressure in the reactor is kept constant by introduction of the gaseous olefinic feedstock until the total volume of liquid produced represents, for example, from 1 to 1000 times the volume of the catalytic solution introduced beforehand. The catalyst is then destroyed by any usual means known to a person skilled in the art and the reaction products and the solvent are then withdrawn and separated.

According to another embodiment, the oligomerization process is performed continuously. The catalytic system, constituted as described above, is injected at the same time as the gaseous olefinic feedstock, preferably ethylene, into a reactor according to the invention, and maintained at the desired temperature. The components of the catalytic system can also be injected separately into the reaction medium. The gaseous olefinic feedstock, preferably gaseous ethylene, is introduced via an inlet valve controlled by the pressure, which keeps the latter constant in the reactor. The reaction mixture is withdrawn by means of a liquid-level-control valve, so as to keep said level constant. The catalyst is destroyed continuously by any usual means known to a person skilled in the art and the products resulting from the reaction, and also the solvent, are then separated out, for example by distillation. The ethylene which has not been converted can be recycled into the reactor. The catalyst residues included in a heavy fraction can be incinerated.

### EXAMPLES

The examples below illustrate the invention without limiting the scope thereof.

### Example 1 (Comparative)

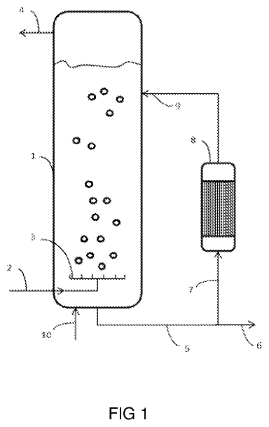

Example 1 illustrates the reference case corresponding to FIG. 1, in which the oligomerization process employs a gas/liquid reactor according to the prior art.

A gas/liquid oligomerization reactor according to the prior art, comprising a reaction chamber of cylindrical shape having a diameter of 1.8 m and a liquid height of 6 m, is employed at a pressure of 7.0 MPa and at a temperature of 120° C.

The catalytic system introduced into the reaction chamber is a chromium-based catalytic system, as described in patent FR 3 019 064, in the presence of cyclohexane as solvent.

Said catalytic system is brought into contact with gaseous ethylene by introduction of said gaseous ethylene into the lower part of said chamber. The effluent is subsequently recovered at the bottom of the reactor.

The volumetric productivity of this reactor is 17 kg of α-olefins produced per hour and per m³ of reaction volume.

The performance qualities of this reactor make it possible to convert 77.4% of the injected ethylene, for a degree of saturation with dissolved ethylene in the liquid phase of 61.0%, and to achieve a selectivity of 83.1% for 1-hexene, for a mass proportion of solvent of 1.6. Said mass proportion of solvent is calculated as the mass ratio of the flow rate of injected solvent to the flow rate of injected gaseous ethylene.

### Example 2 (According to the Invention)

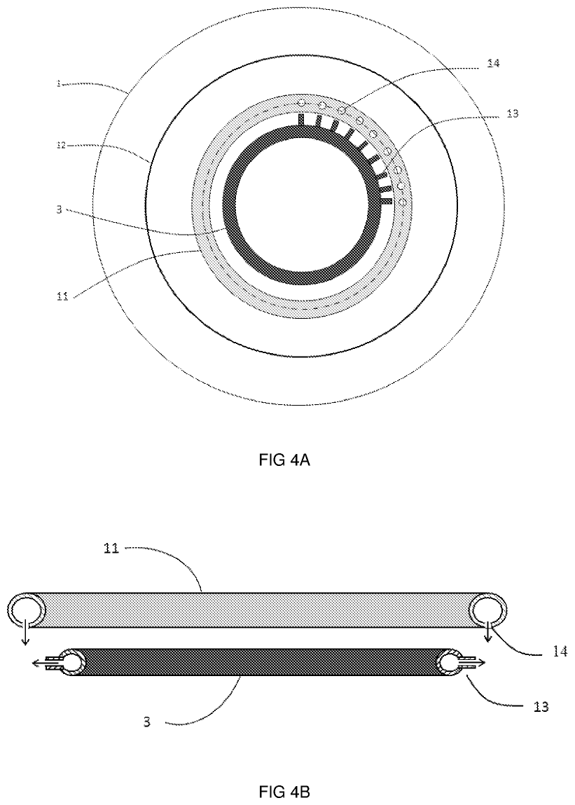

A reactor according to the invention as represented in FIG. 3, having a cylindrical central pipe with a height of 4 m and an inside diameter equal to 0.55 m, is used under the same conditions as in Example 1. The means for introducing ethylene gas (2) and liquid (9), corresponding to the gas injection device (3) and the liquid injection device (11), respectively, are positioned in the top part of said central pipe.

The volumetric productivity of this reactor is 35.7 kg of α-olefin produced per hour and per m³ of reaction volume.

The performance qualities of this reactor make it possible to convert 59.7% of the injected ethylene, for a degree of saturation with dissolved ethylene in the liquid phase of 87.2%, and to achieve a selectivity of 87.1% for the desired α-olefin, for a mass proportion of solvent of 1.6. Said proportion of solvent is calculated as the mass ratio of the flow rate of injected solvent to the flow rate of injected gaseous ethylene.

In Example 2, the reactor according to the invention makes it possible to increase the saturation with ethylene by 26.2%, to increase the selectivity for α-olefin by 4.0% and to multiply the productivity by 2.1, relative to the case according to the prior art of Example 1.

1. A process for the oligomerization of a gaseous olefinic feedstock, said process comprising:
performing oligomerization of the gaseous olefinic feedstock in a gas/liquid reactor at a temperature of between 30 and 200° C., and a pressure of between 0.1 and 10.0 MPa, in the presence of a catalytic system comprising at least one metal precursor,
wherein said gas/liquid reactor has a lower part and an upper part and comprises:
a reactor chamber (1), of elongated shape along a vertical axis, having a lower zone and an upper zone,
a gas injection device (3),
a liquid injection device (11),
a central pipe (12) positioned on the vertical axis inside said chamber in a lower zone of said chamber; said central pipe delimiting a central flow zone that is capable of permitting a descending flow and an outer flow zone that is capable of permitting an ascending flow, and said central pipe having an upper part and a lower part,
in which the gas injection device is positioned in the upper part of said central pipe and the liquid injection device is positioned in the lower zone of the reactor chamber so as to be able to entrain the injected gas in the direction of the lower part of the reactor, from the descending central zone to the ascending outer zone.

2. The process according to claim 1, in which the gas and liquid injection devices are positioned in the upper part of said central pipe so as to entrain the injected gaseous olefinic feedstock in the direction of the lower part of the reactor and from the descending central zone to the ascending outer zone.

3. The process according to claim 2, in which the liquid injection device (11) is positioned above the gas injection device (3).

4. The process according to claim 1, in which the central pipe (12) has a solid wall over the entire height of the central pipe or has apertures over 5% to 10% of the lower part of the height of the central pipe from the lower end aperture.

5. The process according to claim 1, in which the lower part of the central pipe at a lower end aperture exhibits flaring or tapering.

6. The process according to claim 1, in which the central pipe comprises a deflector positioned in the reactor chamber and facing a lower end aperture of the central pipe.

7. The process according to claim 6, in which the deflector is positioned at a distance with the lower aperture of the central pipe corresponding to a distance of between one and two times the equivalent diameter of the central pipe.

8. The process according to claim 6, in which the equivalent diameter of the deflector is at least equal to the equivalent diameter of the central pipe.

9. The process according to claim 1, in which the reactor further comprises a recirculation loop comprising a withdrawing means located at the base of the reactor chamber, a heat exchanger located outside the reactor chamber and an introduction means located on or in the reactor chamber to allow the introduction of a cooled liquid fraction into the reactor chamber.

10. The process according to claim 9, in which the liquid injection device (11) is positioned in the upper part of the central pipe and is connected to the introduction means of the recirculation loop.

11. The process according claim 1, in which the central pipe has an equivalent diameter with a ratio of the equivalent diameter of the central pipe to the inside diameter of the reactor chamber of between 0.2 and 0.9.

12. The process according to claim 1, in which the central pipe has a height with a ratio of the height of the central pipe to the height of the reactor chamber of between 0.2 and 0.8.

13. The process according to claim 1, in which the gas injection device (3) comprises at least one gas injection orifice and the liquid injection device (11) comprises at least one liquid injection orifice, each gas injection orifice being positioned at an orifice of the liquid injection device (11) so that the injection of the liquid can bring about a reduction, by shear, of the size of the bubbles during the injection of the gaseous olefinic feedstock.

14. The process according to claim 13, in which the gas injection orifices and the liquid injection orifices are extended by an injection tube.

15. The process according to claim 1, in which the gaseous olefinic feedstock is chosen from hydrocarbon-based molecules containing between 2 and 6 carbon atoms.

16. The process according to claim 6, in which the equivalent diameter of the deflector is between 0.5 and 2.0 times the equivalent diameter of the central pipe.

17. The process according claim 1, in which the central pipe has an equivalent diameter with a ratio of the equivalent diameter of the central pipe to the inside diameter of the reactor chamber of between 0.3 and 0.8.

18. The process according to claim 1, in which the central pipe has a height with a ratio of the height of the central pipe to the height of the reactor chamber of between 0.3 and 0.7.

19. The process according to claim 1, in which the gaseous olefinic feedstock is chosen from hydrocarbon-based molecules containing between 2 and 4 carbon atoms.

20. The process according to claim 1, in which the gaseous olefinic feedstock is chosen from butenes, propylene, and ethylene, alone or as a mixture.
